(19) United States
(12) Patent Application Publication
(10) Pub. No.: US 2026/0345847 A1
(43) Pub. Date: Nov. 5, 2026

(54) Cobotic System for Sterile Processing and Asset Management

(71) Applicant: Peritas AI Inc., Fort Lauderdale, FL (US)

(72) Inventor: Martin Roche, Fort Lauderdale, FL (US)

(73) Assignee: Peritas AI Inc., Fort Lauderdale, FL (US)

(21) Appl. No.: 19/366,527

(22) Filed: Oct. 23, 2025

Related U.S. Application Data
(63) Continuation-in-part of application No. 19/195,731, filed on Apr. 30, 2025.

(51) Int. Cl. A61L 2/24; A61L 2/28; A61L 103/05

(52) U.S. Cl. CPC A61L 2/24; A61L 2/28; A61L 2103/05; A61L 2202/14; A61L 2202/17

(57) ABSTRACT

A cobotic system is disclosed for automating the sterile processing and asset management of surgical tools within healthcare facilities. The system includes a robotic arm with force and environmental sensors, a tool recognition module for identifying and classifying instruments, and an asset management database for real-time tracking. A tray placement mechanism organizes tools into surgical trays, and an interface coordinates transfer to a sterilization unit. Interchangeable end-effectors enable handling of diverse tool geometries, while a sterile verification module ensures post-sterilization cleanliness. Through integrated automation and sensing, the system improves efficiency, accuracy, and sterility assurance while reducing manual error in surgical tool handling and management.

### CROSS-REFERENCE TO RELATED APPLICATIONS

This patent application is a continuation-in-part of US Patent Application Ser. No. 19/195,731 filed on Apr. 30, 2025, which claims the benefit of priority to U.S. Provisional Patent Application No. 63/640,524, filed on Apr. 30, 2024, the entirety of which is incorporated herein by reference.

### TECHNICAL FIELD

The present invention relates generally to collaborative robotic (cobotic) systems, and more specifically to a cobotic system for sterile processing of surgical tools and instruments.

### BACKGROUND

Sterilization of surgical tools is a critical process in hospitals and surgical centers to prevent infections and maintain high standards of patient care. The process involves cleaning, sterilizing, and organizing various surgical tools before they can be safely used in procedures. Traditionally, this process has been labor-intensive, requiring manual sorting, cleaning, and handling by sterile processing department (SPD) personnel. Manual processing poses risks of human error, including improper sterilization, misplacement or loss of tools, and inconsistency in the arrangement of surgical trays. Additionally, manual handling of tools increases the risk of work-related injuries among SPD staff.

The utilization of robotics in medicine has seen significant advancements in increasing operational efficiency, enhancing accuracy, and reducing human error. However, the application of robotics to the sterile processing of surgical tools has been limited. Existing systems primarily focus on the automation of individual tasks, such as washing or packaging, without an integrated approach to managing and tracking tools throughout the sterilization cycle. There is a gap in current technology for a system capable of automating the entire process of sterile processing and asset management of surgical tools, from identification and sorting to sterilization and storage, while also providing real-time tracking and status updates to users.

Furthermore, traditional systems lack the ability to adapt to the vast variety of surgical tools in terms of shapes, sizes, and material types, which requires sophisticated recognition and handling capabilities. The limitations in existing systems impede their widespread adoption in healthcare facilities that handle a broad range of surgical instruments. Another issue relates to the maintenance of the sterile status of tools, where current methods do not adequately alert staff about maintenance schedules or the expiration of sterile status, leading to potential breaches in sterile protocol. As the complexity of surgical systems and robotics advances, the “Instructions for use” are becoming more complex and require documentation of time, techniques and digitally linked to the Unique Device Identification (UDI) System.

Therefore, there is a need for a comprehensive cobotic system that addresses these limitations by incorporating advanced tool and device recognition, adaptive handling, environmental sensing, and dynamic asset management capabilities. Such a system would improve the efficiency, accuracy, and reliability of the sterile processing department's operations, ensuring the availability of properly sterilized and managed surgical tools for various medical procedures.

### SUMMARY

The present invention relates to a cobotic system and method for the sterile processing and asset management of surgical tools. The system integrates advanced technologies, including robotics, image recognition, machine learning, data management, and user interface technologies, to automate the tasks traditionally performed manually in the SPD of healthcare facilities. By improving the efficiency, accuracy, and reliability of surgical tool sterilization and management, the system addresses current limitations in manual processing, such as human error, work-related injuries to SPD staff, and the lack of real-time tracking and management of surgical tools.

At the core of the system is a cobotic assembly that includes a robotic arm equipped with a tool recognition module and a tray placement mechanism. The robotic arm is designed to manipulate surgical tools safely and precisely, while the tool recognition module utilizes image processing to identify and classify the tools based on predetermined criteria. Information related to the identified tools, including their type, sterilization status, and location within the SPD, is stored and managed in an asset management database, which is communicatively coupled to the tool recognition module.

A user interface allows users to interact with the system, providing inputs and receiving real-time information about the status and management of the surgical tools. The system is capable of arranging tools into surgical trays in a predetermined layout and transporting these trays to a sterilization unit, improving the consistency and reliability of the tool sterilization process.

Advanced features of the system include environmental sensing capabilities for monitoring conditions within the SPD; force sensors on the robotic arm to prevent damage to tools during manipulation; and interchangeable end-effectors to accommodate a variety of surgical tool sizes and geometries. Additionally, the system can select an appropriate sterilization method based on the identified characteristics of the tools, verify the cleanliness of tools post-sterilization, and detect physical deformities or wear in the tools, providing alerts for maintenance or replacement as necessary.

The method embodied by the cobotic system encompasses steps for manipulating, identifying, and classifying surgical tools; managing tool-related information; arranging tools into trays; and sterilizing these tools, all designed to streamline the process of preparing surgical tools for use in medical procedures. By incorporating AI algorithms for tool recognition and offering capabilities for automatic updating of the asset management database both on-site and with various distribution entities, the method ensures high efficiency and adaptability to a broad range of surgical instruments and enables diverse surgical preference cards to be autonomously confirmed.

In another aspect, the disclosure provides a non-transitory computer-readable medium storing instructions that, when executed by one or more processors operatively coupled to the cobotic sterile-processing system—including a robotic arm, tool-recognition module with an image capture device, tray placement mechanism, sterilization module interface, asset-management database, and user interface—cause the processors to: acquire image data of surgical tools and identify and classify the tools; generate and update database records for each tool with at least tool type, location within the SPD, and sterilization status, while maintaining links to the tool’s IFU, UDI data, and any applicable surgeon preference card; determine a predetermined tray layout for a target procedure, detect missing tools, and initiate retrieval of replacements; command the robotic arm, with force-limited motion and selected interchangeable end-effectors, to arrange tools into trays according to the layout; select a sterilization method based on identified tool characteristics and IFU guidance and control the sterilization unit via the interface; verify post-cycle sterility using parametric cycle data and chemical indicators, update the database accordingly, and automatically initiate reprocessing for any tool that fails verification; and present real-time status of tray completion and sterilization together with alerts for maintenance and sterile-status expiration, while recording time-stamped audit data for the foregoing operations.

### BRIEF DESCRIPTION OF DRAWINGS

The foregoing and other objects, aspects, features, and advantages of the disclosure will become more apparent and better understood by referring to the following description taken in conjunction with the accompanying drawings, in which:

FIG. 1 is a schematic diagram of a cobotic system for sterile processing and asset management of surgical tools;

FIG. 2 is a perspective view of a robotic arm equipped with interchangeable end-effectors;

FIG. 3 is a block diagram of a tool recognition module;

FIG. 4 is a flow chart of a process for sterile processing and asset management of surgical tools using the cobotic system;

FIG. 5 is a flow chart of a process for sterile verification;

FIG. 6 is a flow chart of a process for detecting physical deformities, bioburden, or wear in surgical tools by a tool recognition module.

These drawings are provided for illustrative purposes only and should not be construed as limiting the invention to the specific embodiments shown and described herein. Variations and modifications to these embodiments will be apparent to those skilled in the art, and all such variations and modifications are intended to be included within the scope of this disclosure.

### DETAILED DESCRIPTION

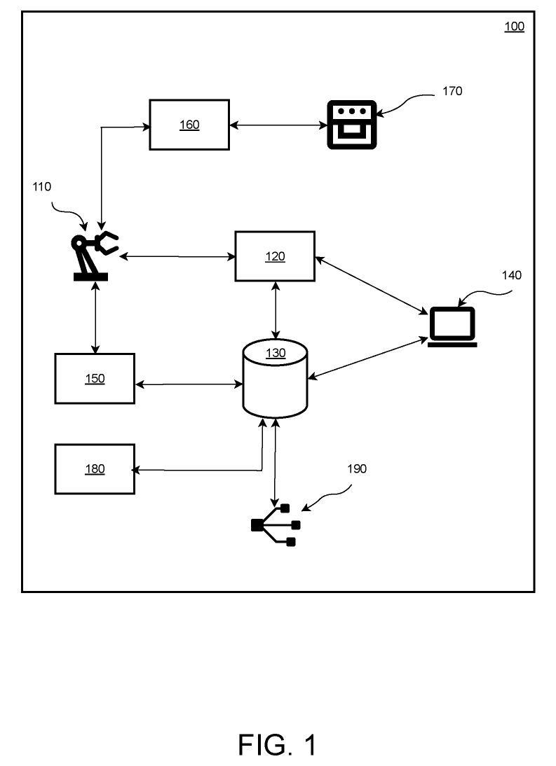

Referring now in detail to the drawings, FIG. 1 illustrates a schematic diagram of the cobotic system 100 for sterile processing and asset management of surgical tools, designed to significantly enhance the efficiency, accuracy, and reliability of surgical tool sterilization and management processes within healthcare facilities.

As depicted in FIG. 1, a robotic arm 110 serves as the primary manipulator for moving, sorting, and accurately positioning surgical tools. This robotic arm 110 is designed to handle the variety of shapes, sizes, and materials of surgical tools, ensuring their precise arrangement in surgical trays and safe transportation to the sterilization unit. The construction of the robotic arm 110 is adapted for delicate interactions with sensitive medical equipment, minimizing the risk of physical damage through the integration of force sensors.

Integrated with the robotic arm 110 is a tool recognition module 120, which is responsible for identifying and classifying the surgical tools. The tool recognition module 120 employs image capture technology coupled with sophisticated image processing software to interpret the visual characteristics of the surgical tools. This module applies predefined criteria to distinguish between different types of tools, thereby facilitating their correct cleaning, assembly and arrangement within trays and ensuring appropriate sterilization protocols are applied.

The asset management database 130, which is in communication with the tool recognition module 130. The database 130 records detailed information about each tool, including its type, current sterilization status, and precise location within the sterile processing department (SPD). This allows for streamlined operations and real-time tracking of surgical tools throughout the sterile processing cycle.

A user interface 140, such as a personal computer, provides a conduit for human interaction with the cobotic system 100. Through this interface 140, users are able to input commands, receive updates, and view the real-time status of surgical tools within the system 100. This high level of access to information empowers SPD personnel to manage operations efficiently, respond swiftly to issues, and ensure that tools are ready and available for surgical procedures.

Attached operatively to the robotic arm 110 is a tray placement module 150, which is designed to precisely arrange the identified surgical tools into surgical trays according to a predetermined layout. This module 150 directs the robotic arm 110 to optimize the organization of tools in order to improve sterilization or to match specific requirements of surgical procedures, thereby facilitating a smoother workflow in the operating room.

Further, the system 100 incorporates a sterilization module interface 160, which mediates the interaction of the robotic arm 110 with a sterilization unit 170 such as an autoclave. After the surgical tools are arranged in trays by the tray placement mechanism 150, the sterilization module 160 enables transportation of the trays into the sterilization unit for sterilization. This module 160 may include, for example, conveyor belts, additional robotic arms, mobile robots, and/or control systems directing the transportation of trays into the sterilization unit.

After the sterilization cycle is complete, a sterile verification module 180 of the system 100 is deployed to verify the sterility of the surgical tools. The sterile verification module 180 includes sensors and indicators designed to detect biological, chemical, or physical markers that signify successful sterilization

The system 100 connects to other perioperative systems outside SPD through a hospital network 190. Connection to the network 190 facilitates coordination of SPD with operating rooms, EHR systems, centralized hospital databases, and so forth to provide visibility to surgical tool sterilization status and location and to generally improve logistical efficiency within a hospital.

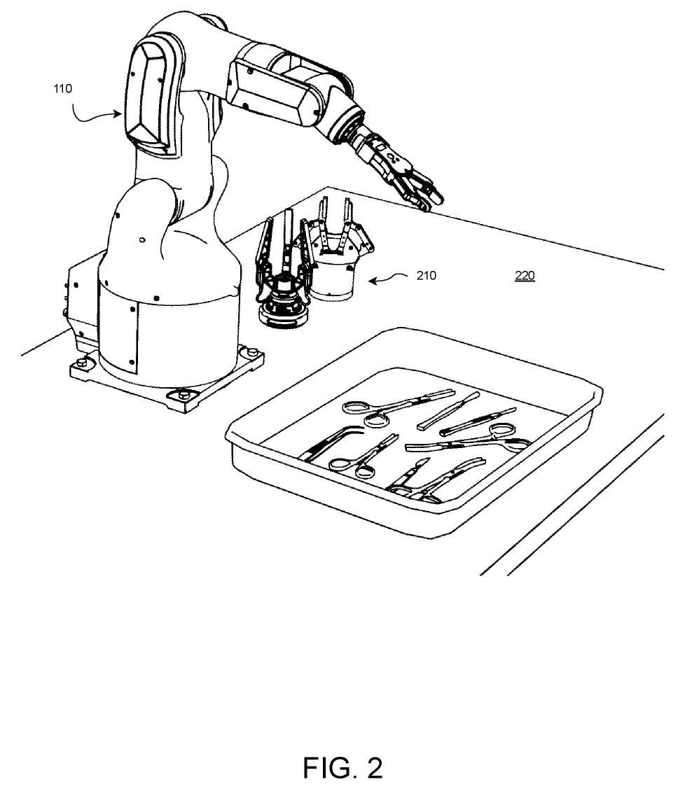

FIG. 2 provides a perspective view of the robotic arm 110, highlighting the inclusion of interchangeable end-effectors 210. These end-effectors 210 enable robotic arm 110 to adapt to and manipulate a wide variety of surgical tool shapes, sizes, and materials with precision and care.

The robotic arm 110, depicted in FIG. 2, demonstrates a flexible design that accommodates the complex needs of SPDs. Each end-effector 210 can be easily attached or detached from the robotic arm 110, allowing for quick adaptation to different tasks without the need for manual reconfiguration or prolonged downtime. This modular design supports the system's versatility, facilitating the handling of both delicate instruments and bulkier surgical equipment.

Another aspect of the interchangeable end-effectors 210 is their integration with force sensors. These sensors provide real-time feedback on the amount of force being applied during the manipulation of surgical tools. This capability enables secure handling of tools while minimizing the risk of damage during sorting, arrangement, and transportation processes. The force sensors are calibrated to account for differences in structure and material composition of different surgical tools, allowing the robotic arm 110 to adjust its grip and movement dynamically by leveraging the tool recognition module 120.

Each end-effector 210 is designed with a specific set of tasks in mind, ranging from gripping, lifting, to gentle repositioning. Some end-effectors may feature soft, flexible surfaces for handling delicate items, while others might include more specialized configurations for securely gripping irregularly shaped or bulky items. The design and material selection for each end-effector consider the need for sterility and compatibility with various sterilization processes, ensuring that the end-effectors themselves can be easily and effectively sterilized as needed. Specialized end effectors with combined pressure and camera systems, tactile coverings and magnetic elements can be utilized based on instrument recognition and handling requirements.

The robotic arm 110 is anchored to a stable base 220 such as a workbench, which provides the necessary support for the arm's range of motion. This base 220 is engineered to maintain stability even when the arm extends fully or operates at high speeds, thereby ensuring precision and safety in all operational scenarios. In another embodiment the base 220 is a mobile base that supports autonomous movement of the cobotic system 100 in the SPD. In an embodiment the mobile base is part of a bipedal has a humanoid form factor.

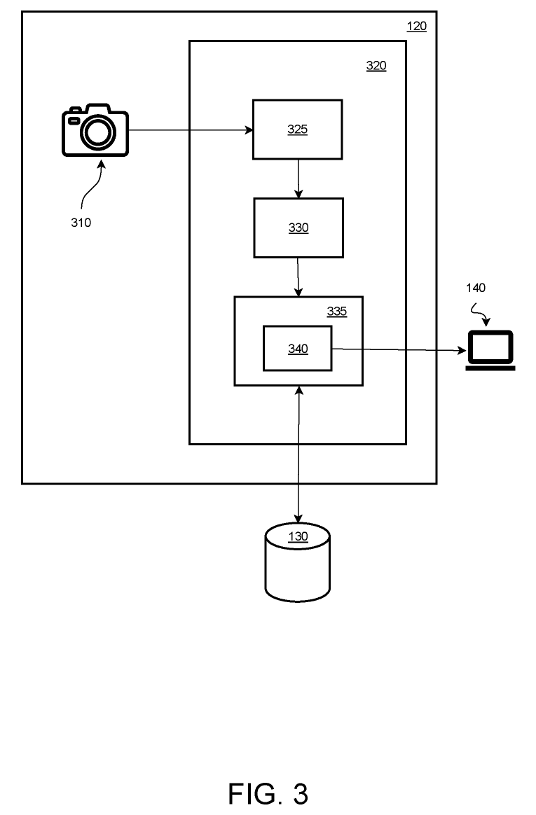

FIG. 3 illustrates the tool recognition module 120 in greater detail. The tool recognition module 120 enables the cobotic system 100 to accurately identify and classify a wide variety of surgical tools based on their physical characteristics, intended functions, surgical preference cards and instructions for use.

The tool recognition module 120 comprises two primary components: an image capture device 310 and a processing unit 320. The image capture device 310 is strategically positioned to obtain high-resolution images of the surgical tools either individually or within the arrangement on the trays. This device may incorporate various imaging technologies, including mono or stereo digital cameras, 3D scanners, LiDAR, or a combination thereof, to capture detailed visual data from multiple angles, to obtain broad coverage of the tool's features.

Once an image is captured, it is transmitted to the processing unit 320, which hosts image processing software. The software executes a series of stages, beginning with preprocessing 325, in which the incoming image data is decoded, resized, and normalized to a standardized format suitable for model input. Decoding converts the compressed or encoded image data into raw pixel arrays, resizing scales the image to match the input resolution required by the neural network, and normalization adjusts pixel intensity values, for example to a 0 to 1 or -1 to 1 range, to enhance model stability and inference accuracy.

Following preprocessing, the system proceeds to model execution 330, where the processed image data is analyzed using one or more machine-learning models. In certain implementations, the model execution 330 utilizes a convolutional neural network (CNN) configured for classification, feature extraction, or object localization within the field of view. In alternative or complementary embodiments, a diffusion-based neural network may be employed to perform probabilistic reconstruction and classification, iteratively refining latent image representations to enhance detection confidence and segmentation precision. These approaches enable robust identification of surgical tools or other objects across variable lighting and occlusion conditions.

The results of the model execution are then passed to a post-processing module 335, which interprets the raw model outputs to generate a structured result. Depending on the configuration, the post-processing module 335 may output a bounding box around each detected object, a segmentation mask delineating object boundaries, and an associated class label identifying the detected item. These outputs are transmitted to downstream systems for visualization, further decision-making, or storage in the asset management database 130.

In an embodiment the post-processing module 335 uses the outcome of the processing to compare against a predefined set of criteria stored within database 130. These criteria include physical dimensions, shapes, surface textures, and any specific markings or features unique to certain tool types. Upon successful identification and classification, the tool recognition module 120 updates the database 130 with the relevant information about each tool, such as its type, expected sterilization parameters, and designated location within the surgical tray layout.

Furthermore, the tool recognition module 120 is engineered to operate in real-time, offering immediate feedback to the robotic arm 110. This capability facilitates the dynamic adjustment of system’s 100 actions based on the rapid identification of tools, optimizing the sorting and arrangement processes with minimal delay. In an embodiment tool recognition module identifies surgical tools in images from image capture device at a rate between 10 and 60 Hz.

An additional feature of the tool recognition module 120 involves quality assurance checks. A quality assurance module 340 incorporated into the post-processing module 335 is capable of detecting signs of physical wear, contamination, or damage that may compromise their safe use. The module can issue alerts or updates through the user interface 140 regarding any tools that require maintenance, repair, or replacement, thereby contributing to the overall reliability and safety of the surgical tool management process.

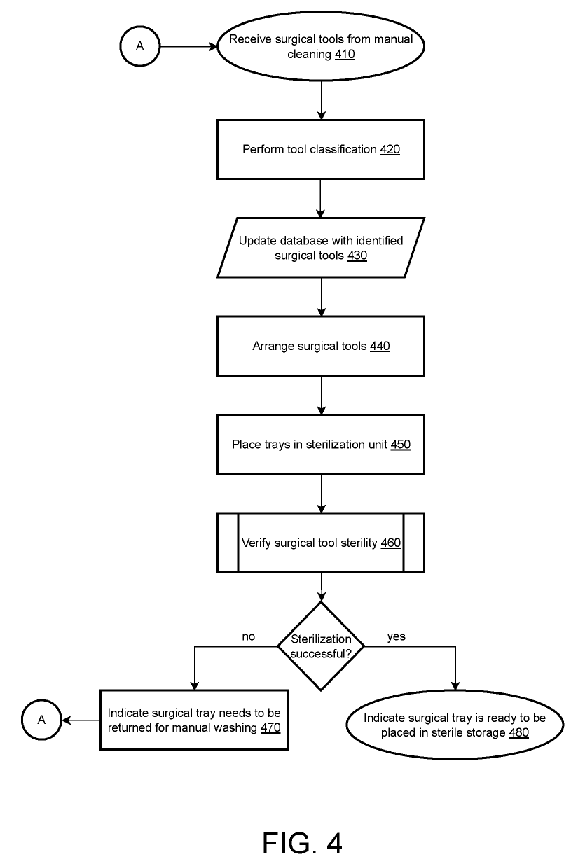

FIG. 4 is a flow chart detailing a process for sterile processing and asset management of surgical tools using the cobotic system 100. Surgical tools are generally pre-cleaned at the point of use and manually cleaned upon arrival at SPD. The process detailed in FIG. 4 includes the steps following manual cleaning through final sterilization and verification.

At the commencement of the process, step 410 involves the robotic arm 110 receiving surgical tools from manual cleaning that require sterilization. This initial step marks the beginning of the automated process where tools are placed within the system's operating area, either manually by staff or through an integrated delivery mechanism. The system is designed to handle tools individually or in bulk, depending on the workflow requirements and the configuration of incoming tools.

Following the reception of tools, step 420 activates the tool recognition module 120, initiating the identification and classification sequence for each surgical tool. During this phase, the image capture device 310 of the tool recognition module 120 captures high-resolution images of the tools. These images are then analyzed by the processing unit 320, employing machine learning algorithms to accurately identify and classify tools based on their physical characteristics, including size, shape, and any distinguishing features. This step assists the SPD technician and cobotic system in determining the appropriate handling and sterilization procedures for each tool type.

Step 430 involves the updating of the asset management database 130 with information obtained from the tool identification and classification process. This database serves as a repository, recording details such as tool type, necessary sterilization methods, and the precise location of tools within the SPD. The real-time updating of the database enables accurate tracking and management of surgical tools, convenient retrieval, and a high level of operational efficiency within the SPD.

In step 440, the cobotic system 100, through the functionality of the tray placement mechanism 150, arranges the identified surgical tools into designated surgical trays. This step is guided by a predetermined layout specific to a given tray configuration. The accuracy and consistency provided by automated arrangement minimizes the potential for human error, ensuring that surgical teams receive complete trays for each procedure.

Following the arrangement of tools into trays, step 450 involves the transport of these trays to the sterilization unit 170 via the sterilization module 160. The sterilization module 160 oversees this process, automating the process of blue wrapping the trays and ensuring secure transfer of the prepared trays into the sterilization unit 170. In another embodiment the sterilization module 160

In step 460, which can occur concurrently with the sterilization process or thereafter, engages the sterile verification module 180 to verify the sterility of the surgical tools post-sterilization. This step employs collected sterilization indicator data compared with predefined sterilization criteria to assess whether the tools in the surgical tray under consideration meet the hospital's or regulatory standards for sterility.

In step 470, if the surgical tools fail the verification process the database is updated to flag the surgical tools as requiring reprocessing and the module 180 indicates that the surgical tray has failed and needs to be returned for manual washing in step 490. This indication of failed verification could be, for example, the illumination of red LED or a negative prompt on the user interface 140. After manual washing, the sterilization process is repeated, returning to step 410

In step 480, if the surgical tools pass the verification process the database is updated to mark the surgical tools as “sterile” and ready to use in surgical procedures and the module 180 indicates that the surgical tray containing the sterilized surgical tools is ready to be placed in sterile storage. This indication of successful verification could be, for example, the illumination of green LED or a positive prompt on the user interface 140.

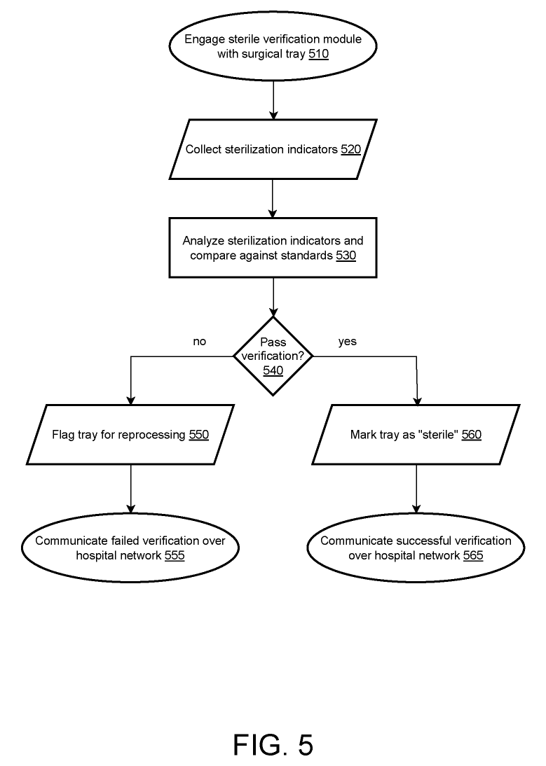

FIG. 5 depicts a flow chart representing a sub-process for sterile verification of surgical tools identified as step 460 in FIG. 4. This flow chart outlines the steps involved in verifying the sterility of the surgical tools post-sterilization, employing advanced sensing and validation technologies to ensure tools meet the required sterility standards before they are deemed ready for surgical use.

Step 510 initiates the sterile verification process, where the sterile verification module 180 is engaged with the tray sterilized tools and trays to begin assessment of the surgical tools' adherence to sterility protocols. This module is equipped with sensors and indicators designed to detect biological, chemical, or physical markers that signify successful sterilization. For instance, this could involve the detection of color changes in chemical indicators or the analysis of biological samples to confirm the absence of microbial contamination.

In step 520, the sterilization verification module 180 collects sterilization indicators from the sensors in communication therewith. This can include sensors that are active during the sterilization process, such as temperature and pressure sensor, and/or sensors that measure characteristics of the surgical tray after the sterilization cycle, without disturbing the blue wrap, such as a chemical integrator, residual bioburden test, or wet load indicator.

In step 530, the sterilization verification module 180 analyzes the data collected in step 520 and compares it against hospital and/or regulatory sterility standards to determine whether the surgical tools are ready for use. This determination is made in step 540 for each tray. If the verification process identifies any discrepancies or failures in meeting the established sterilization criteria, the system proceeds to step 550. Otherwise, if all criteria are satisfactorily met, the process advances to step 560.

Step 550 addresses the handling of tools or trays that did not meet sterility standards. In this step, the system flags these tools or trays for reprocessing, automatically updating the asset management database 130 to reflect their current status and initializing a re-sterilization cycle. This ensures tools do not advance to surgical use without first meeting necessary sterility requirements.

Following the updating of the database in step 550, the sterile verification module communicates, in step 555, the negative outcome to the user interface 140 within SPD as well over the network 190, providing real-time information to healthcare staff throughout the hospital regarding the reprocessing of the surgical tools and potential delay associated with their availability. This enables surgical teams that may have been relying on the availability of these tool to promptly plan and effect a contingency and to reprioritize, if necessary, the sterilization queue.

Step 560 represents the successful completion of the sterile verification process for tools and trays that have met all sterilization criteria. At this juncture, the system updates the asset management database 130 with a sterilization confirmation for each tool and tray, marking them as "sterile" and ready for use in surgical procedures. This update includes detailed records of the sterilization process, such as the method used, environmental conditions met, and the results of direct and/or indirect sterility indicators.

Following the updating of the database in step 560, the sterile verification module communicates, in step 565, the positive outcome to the user interface 140 within SPD as well over the network 190, providing real-time information to healthcare staff throughout the hospital regarding the availability and sterility status of the surgical tools. This ensures that surgical teams are promptly and accurately informed of the readiness of tools for upcoming procedures, facilitating efficient planning and preparation for surgeries.

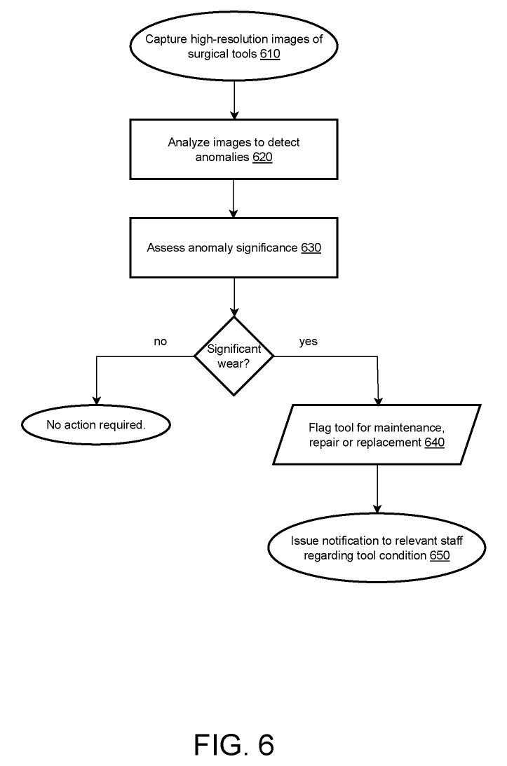

FIG. 6 depicts a process for detecting physical deformities, contamination or excessive wear in surgical tools by utilizing the quality assurance module 340 within the tool recognition module 120. This process facilitates verification of the physical integrity and operational readiness of the surgical tools.

Step 610 initiates the process with the tool recognition module 120 capturing high-resolution images of the surgical tools, either during the initial identification phase or as a separate quality assurance procedure. This step facilitates assessing the condition of the surgical tools and identifying any signs of wear, damage, or deformation that could compromise their effectiveness during surgical procedures.

Following image capture, step 620 involves the processing unit 320 analyzing the images to detect anomalies indicative of physical wear or damage. The analysis leverages advanced image processing algorithms and machine learning techniques to accurately identify discrepancies between the current state of a tool and its expected condition, based on a database of tool images and criteria for physical integrity. This step benefits from a dataset enriched with various examples of tool wear and damage, enabling the system to distinguish between normal variations in tool appearance and genuine signs of concern.

In step 630, the system applies predefined criteria to determine the significance of detected anomalies. These criteria consider factors such as the extent of wear, the location of damage, and the specific function of the tool, assessing the potential impact on both tool performance and patient safety. Considering, for example, a scalpel, the scalpel blade would be identified as a critical region and even minimal wear or notching on the blade would cause the module 340 to flag the scalpel for replacement, whereas the same wear or notching on the handle may register as within acceptable limits of wear. The assessment in step 630 helps prioritize issues based on their urgency and the need for intervention, ensuring that tools requiring immediate attention are promptly identified.

If significant wear or damage is detected, as determined in step 630, the tool is tagged within the asset management database 130 for maintenance, repair, or replacement in step 640. This step involves updating the tool's record with details about the detected issues, recommendations for action, and potentially removing the tool from active circulation within the SPD. This tagging ensures that compromised tools are not inadvertently used in surgical procedures, maintaining high standards for safety and effectiveness.

Step 650 involves issuing alerts or notifications through the user interface 140 and/or network 190 regarding the tools flagged for attention. These alerts inform the relevant personnel about the condition of the tools, providing specifics on the issues detected and suggesting next steps, whether it be servicing or procuring replacements. By proactively notifying staff, the system facilitates timely action to address tool integrity, thereby contributing to the efficient operation of the SPD and the overall safety of surgical interventions.

As used herein, “surgical tools” refers to surgical tools, surgical devices, surgical instruments, and similar implements used in surgery; and “hospitals” refers to hospitals, ambulatory surgery centers, and similar healthcare facilities where surgery is performed.

While specific embodiments have been described to enable practice of the disclosed subject matter, they are illustrative and not limiting. Variations, modifications, substitutions, and combinations of the features described can be made by those skilled in the art without departing from the scope of this disclosure. Features described with respect to one embodiment may be used with, or substituted for, features of another. Unless expressly stated otherwise, the terms “comprising,” “including,” and “having” are used in an open, non‑limiting sense; the singular forms “a,” “an,” and “the” include the plural forms; and stated ranges include their endpoints and all subranges. Unless the context clearly indicates otherwise, “and/or” means one or more of the listed items (e.g., “A and/or B” encompasses A alone, B alone, or both A and B); “or” is inclusive (i.e., “A or B” means A, B, or both); and “and” is conjunctive. Headings and ordering are for convenience and do not limit the description. The scope of the claimed subject matter is defined solely by the claims that follow and their legal equivalents.

What is claimed is:

1. A cobotic system for sterile processing and asset management of surgical tools, comprising:
a robotic arm configured to manipulate the surgical tools;
a tool recognition module integrated with the robotic arm and configured to identify and classify each surgical tool based on predetermined criteria;
an asset management database communicatively coupled to the tool recognition module, the database storing information for each surgical tool including at least the type, sterilization status, and location within a sterile processing department;
a user interface configured to receive input from and display information to a user regarding the status and management of the surgical tools;
a tray placement mechanism operatively connected to the robotic arm and configured to arrange the identified surgical tools into surgical trays based on a predetermined layout;
a sterilization module interface operatively connected to the robotic arm and configured to transport each surgical tray with the arranged tools into a sterilization unit;
wherein the cobotic system is configured to detect any surgical tool that is missing from the set of tools required by a predetermined tray layout, and to initiate retrieval of a replacement for each such missing tool; and
wherein the cobotic system is connected to a database containing instructions for use (IFU) for each surgical tool and is configured to follow each tool’s IFU guidelines during sterilization.

2. The cobotic system of claim 1, wherein the tool recognition module comprises:
an image capture device for capturing images of the surgical tools; and
a processor executing image processing software to analyze the captured images and identify the surgical tools based on the images.

3. The cobotic system of claim 1, wherein the asset management database is further configured to store surgeon preference card confirmation and track the sterilization cycle of each surgical tool, including sterilization dates and methods.

4. The cobotic system of claim 1, wherein the user interface is configured to provide alerts regarding maintenance schedules for the surgical tools and the expiration of each tool’s sterile status.

5. The cobotic system of claim 1, further comprising an environmental sensing module capable of detecting environmental conditions within the sterile processing department, including temperature and humidity levels.

6. The cobotic system of claim 1, wherein the robotic arm includes a force sensor, a camera, a magnetic sensor, or a haptic sensor thereof configured to prevent damage to the surgical tools while ensuring their secure manipulation.

7. The cobotic system of claim 1, wherein the sterilization module interface is configured to select a sterilization method for the surgical tools from among a plurality of available sterilization methods, based on the type and material of each identified surgical tool.

8. The cobotic system of claim 1, wherein the robotic arm is equipped with interchangeable end-effectors to accommodate manipulation of a variety of surgical tools of different sizes and geometries.

9. The cobotic system of claim 1, further comprising a sterile verification module connected to the robotic arm, the sterile verification module being configured to verify the cleanliness of the surgical tools after sterilization according to predefined cleanliness criteria.

10. The cobotic system of claim 1, wherein the tool recognition module is further configured to detect any physical deformities or wear in the surgical tools and to alert the user via the user interface if replacement or repair is necessary.

11. A method for sterile processing and asset management of surgical tools using a cobotic system, comprising the steps of:
manipulating the surgical tools using a robotic arm;
capturing images of the surgical tools via an integrated tool recognition module;
analyzing the captured images with image processing software to identify the surgical tools based on predetermined criteria;
storing information related to the identified surgical tools in an asset management database, the information including at least the type, sterilization status, and location within a sterile processing department;
displaying information to a user and receiving input from the user via a user interface regarding the status and management of the surgical tools;
arranging the identified surgical tools into surgical trays based on a predetermined layout using a tray placement mechanism operatively connected to the robotic arm;
detecting any surgical tool that is required by the predetermined layout but is missing, and obtaining a replacement tool for each such missing tool;
transporting the surgical trays with the arranged tools into a sterilization unit via a sterilization module interface; and
retrieving sterilization instructions for the surgical tools from a database and controlling the sterilization unit to sterilize the surgical tools in accordance with the retrieved instructions.

12. The method of claim 11, further comprising the step of tracking the sterilization cycle of each surgical tool, including sterilization dates and methods, within the asset management database.

13. The method of claim 11, wherein the image processing software uses AI to identify and classify the surgical tools.

14. The method of claim 11, further comprising the step of detecting conditions within the sterile processing department, including temperature and humidity levels, using an environmental sensing module.

15. The method of claim 11, further comprising the step of selecting a cleaning, assembly or sterilization method, via the sterilization module interface, based on the type and material of the identified surgical tools from among a plurality of available sterilization methods.

16. The method of claim 11, wherein analyzing the captured images to identify and classify the surgical tools includes utilizing an AI algorithm trained on a dataset of various surgical tools and their respective classifications.

17. The method of claim 11, further comprising the step of verifying, using the sterile verification module, the cleanliness of the surgical tools after sterilization, and updating the sterilization status of each surgical tool in the asset management database based on the verification results.

18. The method of claim 11, further comprising the step of detecting any physical deformities or wear in the surgical tools using the tool recognition module, and providing an alert to the user via the user interface if a replacement or repair is necessary.

19. The method of claim 11, where the asset management database is connected to a hospital network to provide users or equipment outside the sterile processing department with information relating to the sterilization status and location of the identified surgical tools.

20. A non‑transitory computer‑readable medium storing instructions that, when executed by one or more processors operatively coupled to a cobotic sterile‑processing system comprising a robotic arm, a tool recognition module including an image capture device, a tray placement mechanism, a sterilization module interface, an asset management database, and a user interface, cause the processors to perform operations comprising:
acquiring image data for a plurality of surgical tools and identifying and classifying each surgical tool in view of predetermined criteria;
writing, for each identified surgical tool, a record to the asset management database including at least a tool type, a location within a sterile processing department, and a sterilization status, and linking the record to corresponding instructions for use, a unique device identifier, and a surgeon preference card for a target procedure;
determining, for the target procedure, a predetermined tray layout and detecting any surgical tool that is required by the tray layout but is missing, and in response initiating retrieval of a replacement for each such missing tool;
commanding the robotic arm, with force‑limited motion and a selected interchangeable end‑effector, to arrange the identified surgical tools into a surgical tray in accordance with the predetermined tray layout;
selecting, by reference to the instructions for use and identified tool materials, a sterilization method from among a plurality of available sterilization methods, and controlling, via the sterilization module interface, a sterilization unit to execute a sterilization cycle using the selected method;
verifying post‑cycle sterility by evaluating parametric data from the sterilization cycle or chemical indicators associated with the surgical tray, and based on the verification, updating the sterilization status for each surgical tool in the asset management database and automatically initiating reprocessing for each surgical tool that fails verification; and
presenting, via the user interface, real‑time status of tray completion and sterilization, together with alerts regarding maintenance of surgical tools and expiration of sterile status, and recording time‑stamped audit data for the foregoing operations.
